SEQUENCE LISTING (8 sequences)

SEQ ID NO: 1 - DNA, 23 bp, Artificial Sequence
  PCR upstream primer
gatgagttcgtgtccgtacaact

SEQ ID NO: 2 - DNA, 25 bp, Artificial Sequence
  PCR downstream primer
ggttatcgaaatcagccacagcgcc

SEQ ID NO: 3 - DNA, 25 bp, Artificial Sequence
  PCR upstream primer
gccattctcaccggattcagtcgtc

SEQ ID NO: 4 - DNA, 22 bp, Artificial Sequence
  PCR downstream primer
agccgccgtcccgtcaagtcag

SEQ ID NO: 5 - DNA, 27 bp, Artificial Sequence
  PCR upstream primer
gccctcgagatggtgaatccgggcagc

SEQ ID NO: 6 - DNA, 27 bp, Artificial Sequence
  PCR downstream primer
gccctcgagtcagcagaccttctggtc

SEQ ID NO: 7 - DNA, 19 bp, Artificial Sequence
  PCR upstream primer
ggaattcatgctgttagaa

SEQ ID NO: 8 - DNA, 29 bp, Artificial Sequence
  PCR downstream primer
cgcggatccccgaagcgcttaaagaagtc

(19) United States
(12) Patent Application Publication
(10) Pub. No.: US 2011/0177563 A1
(43) Pub. Date: Jul. 21, 2011

(54) HIGH THROUGHPUT DEVICE FOR PERFORMING CONTINUOUS-FLOW REACTIONS

(75) Inventors: Jong Hoon HAHN, Pohang (KR); Nokyoung PARK, Pohang (KR); Kwanseop LIM, Pohang (KR)

(73) Assignees: POSTECH FOUNDATION, Pohang (KR); BIONEER CORPORATION, Daejeon (KR)

(21) Appl. No.: 13/025,741

(22) Filed: Feb. 11, 2011

Related U.S. Application Data
(63) Continuation of application No. 10/588,159, filed on Aug. 1, 2006, now abandoned.

(51) Int. Cl. C12P 19/34; C12M 1/00; C12M 1/34

(52) U.S. Cl. 435/91.2; 435/293.1; 435/293.2; 435/287.2

(57) ABSTRACT

A high-throughput device is structured to perform a continuous-flow reaction, e.g., a polymerase chain reaction (PCR) requiring repetitive temperature control in a timely fashion.

This application is a continuation application of U.S. application Ser. No. 10/588,159, filed Aug. 1, 2006, which claims the benefit of International Application No. PCT/KR2004/000194, filed on Feb. 3, 2004, the disclosures of which are incorporated herein in their entireties by reference.

### FIELD OF THE INVENTION

The present invention relates to a high-throughput device for performing continuous-flow reactions and, more particularly, to a high-throughput device for performing continuous-flow reactions, comprising solid heating blocks and capillary tubes, which performs reactions requiring repetitive temperature controls and reactions in a timely fashion, such as a polymerase chain reaction.

### BACKGROUND OF THE INVENTION

DNA can be artificially replicated in vitro by a DNA replication technology named polymerase chain reaction (PCR) developed by Mullis et al. in 1983. The PCR is a reaction using an enzyme and requires repetitive temperature control at two or three temperature ranges depending on the type of the enzyme.

Generally, the PCR can be made by the following three different steps: a melting step in which a double-stranded template DNA to be replicated denatures into two single-stranded DNA; an annealing step in which primers bind to the denatured single-stranded DNA to designate a place where the reaction starts and assist the initiation of enzyme reaction; and an extension step in which DNA is replicated from the position where the primers bind to produce complete double-stranded DNA. Upon completion of these three steps of the PCR, the final amount of DNA is doubled. That is, if the PCR is repeatedly performed in n times, the final amount of DNA becomes 2ⁿ times. In conventional PCR reactor systems, temperature-adjustable heating blocks are used and are designed to accommodate PCR containers. After the PCR containers are inserted into the heating blocks, PCR is performed by repetitive temperature controls at regular intervals.

In particular, one of the most important factors in performing the PCR successfully is the temperature control. Especially, the temperature control during the annealing step among the three steps of PCR is very important since the improper temperature control at the annealing step causes a decrease in amplification efficiency or specificity, giving a poor PCR yield. Further, monitoring promptly and continuously the course of the PCR in real-time is very important to improve the PCR efficiency during DNA amplification, considering that it takes about several hours until PCR is completed.

Following the introduction of lab-on-a-chip concept for PCR in 1990s, the development of different techniques for PCR is being improved (Northrup et al., Anal. Chem. 1998, 70: 918-922; Waters et al., Anal. Chem. 1998, 70: 5172-5176; Cheng et al., Nucleic Acids Res. 1996, 24: 380-385). Especially, the development of methods and devices for performing continuous-flow PCR has been instrumental for the successful analysis of various kinds of DNA on a single chip.

For instance, Manz et al. developed a device performing continuous-flow PCR in 1998 (Manz et al., Science, 1998, 280: 1046-1048). They linearly arranged three temperature-adjustable copper blocks for the sequential control of melting, extension, and annealing reaction step of PCR process. The PCR product formed was allowed to flow through micro channels on a glass substrate which was mounted over the copper blocks. The temperature of the three different reaction zones have maintained rather smoothly at 95° C.->72° C.->60° C. However, the inherent problem in this arrangement is that the denatured single-stranded DNA sample is passed through the extension reaction chamber before the annealing reaction chamber which reduces substantially the accuracy of the reaction.

Quake et al. tried to solve the above problem by employing a circular arrangement of heating blocks in the sequence of melting, annealing, and extension, instead of the linear arrangement (Quake et al., Electrophoresis, 2002, 23: 1531-1536).

Roeraade et al. also developed a device for performing continuous-flow PCR within a capillary tube using circular water baths controlled at different temperatures. The device was prepared by making several small holes on the wall of the water baths and winding a Teflon tube around the water baths through the holes (Roeraade et al., J. Anal. Chem. 2003, 75: 1-7). It required, however, an agitation device for pumping water at a constant rate for controlling the temperature and water evaporation as well. This requirement makes inconvenience to the development of miniaturised portable PCR device.

### SUMMARY OF THE INVENTION

Accordingly, it is an object of the present invention to provide a high-throughput device for performing continuous-flow reactions comprising solid heating blocks and capillary tubes, which performs repetitive temperature controls and repetitive reactions in a timely fashion, such as a polymerase chain reaction.

It is another object of the present invention to provide a high-throughput method of performing a continuous-flow nucleic acid amplification by using the high-throughput device for performing continuous-flow reactions.

### BRIEF DESCRIPTION OF THE DRAWINGS

The above and other objects and features of the present invention will become apparent from the following description of the invention, when taken in conjunction with the accompanying drawings, in which:

FIG. 1a and FIG. 1b illustrate an outlook of a high-throughput device for performing continuous-flow reactions in accordance with a first preferred embodiment of the present invention;

FIG. 2a and FIG. 2b represent a schematic view and a photograph of a device in accordance with a second preferred embodiment of the present invention, respectively;

FIG. 3a shows a scheme for preparing a heating block-insulating block assembly around which a capillary tube is wound to prepare a high-throughput multiplex device for performing continuous-flow reactions of the present invention;

FIG. 3b presents a plan view of an exemplary multiplex device for performing continuous-flow reactions of the present invention;

FIG. 3c offers a front view of an exemplary multiplex device for performing continuous-flow reactions of the present invention;

FIG. 3d depicts a photograph of a multiplex device for performing continuous-flow reactions prepared in accordance with a third preferred embodiment of the present invention;

FIG. 3e pictorializes a photograph of a multiplex device for performing continuous-flow reactions prepared by winding a capillary tube around the device of FIG. 3d and equipping it with a heater and a sensor;

FIG. 4 describes an exemplary device for detecting real-time reaction, where a device for performing continuous-flow reactions is equipped with an apparatus for real-time detection;

FIG. 5 explains a result of gel electrophoresis identifying DNA amplification after performing PCR by a device for performing continuous-flow reactions of the present invention; and

FIG. 6 accords a result of gel electrophoresis identifying DNA amplification after performing sequential PCRs having different compositions.

### DETAILED DESCRIPTION OF THE INVENTION

The present invention provides a high-throughput device for performing a continuous-flow reaction comprising: (1) at least two solid heating blocks controlled at different temperatures; and (2) at least one capillary tube having a first open end for fluid inlet and a second open end for fluid outlet to permit continuous flow of a fluid from the first open end to the second open end, wherein the capillary tube contacts the heating blocks sequentially or repetitively.

The present invention also provides a high-throughput device for performing a continuous-flow reaction, further comprising at least one insulating block contacting the heating blocks and arranged to prevent the heating blocks from contacting each other.

Further, the present invention provides a high-throughput method of performing a continuous-flow nucleic acid amplification, comprising the steps of: (a) injecting at least one PCR mixture into the first open end of the capillary tube in the aforementioned device; and (b) controlling a flow rate of the polymerase chain reaction mixture at an appropriate speed and collecting a polymerase chain reaction product discharged from the second open end.

In the device of the present invention, each heating block functions to transfer heat to specific parts of the capillary tube and the temperature of the heating block can be controlled to different temperature ranges by a heater and a temperature sensor. The heater and the temperature sensor may be attached to the heating block or inserted into holes formed in the heating block.

There is no limitation as to the heating block materials, as long as they have high heat conductivity. Specifically, metals such as copper, iron, aluminum, brass, gold, silver, and platinum are preferred, and polymer having high heat conductivity can be also used.

The insulating block functions to prevent heat transfer between the heating blocks. Likewise, there is no limitation as to the insulating block materials, as long as they have high insulating property. It is preferred to use bakelite or acrylic polymer resin.

The heating blocks and insulating blocks may be prepared in the shape of a cylinder, an oval, a square, and the like, but there is no limitation as to their shape.

The capillary tube functions as a fluid passage and reaction space and it has a first open end for fluid inlet and a second open end for fluid outlet to permit continuous flow of a fluid from the first open end to the second open end. There is no limitation as to the capillary tube as long as it is commercially available. The capillary tube can be made of various materials, such as glass and polymer. Preferably, the capillary tube may be made of a material selected from the group consisting of glass, fused silica, polytetrafluoroethylene (PTFE; trademark name: Teflon), and polyethylene, which have resistance to heat above 100° C. and to the permeation of an aqueous solution or organic solvent.

Especially, in case the capillary tube is made of glass, it is preferred that the outer wall of the capillary tube is coated with polyimide or PTFE to prevent the breakage of the capillary tube in the process of preparing the device in accordance with the present invention, for example, in the process of winding the capillary tube around the heating blocks. On the other hand, in case the capillary tube is used in a device for detecting real-time reaction, it is preferred to use a transparent capillary tube through which light can pass. If the outer wall of the capillary tube is coated with polyimide, it is preferable to remove the coating on the parts of the tube through which light is irradiated and fluorescent light emits.

Moreover, the inner wall of the capillary tube is preferably silanized to prevent the adsorption of DNA or protein. The silanization may be performed in accordance with well-known methods in the art. Preferably, materials having hydrophobic groups after reacting with the surface of the glass are used for the silanization. More preferably, at least one material selected from the group consisting of trimethylchlorosilane, dimethyldichlorosilane, methyltrichlorosilane, trimethylmethoxysilane, dimethyldimethoxysilane, and methyltrimethoxysilane is used.

The diameter and length of the capillary tube can vary with the type of the fluid flowing inside the tube and that of the reaction to be performed. The inner diameter of the capillary tube may preferably lie in the range of 10 to 300 μm, and the outer diameter of the capillary tube may be preferably in the range of 50 to 500 μm. It is preferable for the length of the capillary tube to be in the range of 0.5 m to 5 m.

In the device of the present invention, the capillary tube can contact heating blocks controlled at different temperatures if it is wound around the heating blocks. One of the methods for winding the capillary tube around the heating blocks is to form a helical groove of a predetermined size and interval on the outer surface of the heating blocks and to fit the capillary tube into the helical groove. The size and interval of the helical groove may vary with the diameter of the capillary tube to be fitted into. It is preferred for the helical groove to have a depth ranging approximately from 100 μm to 500 μm, a width ranging from 100 μm to 500 μm, and an interval ranging from 100 μm to 1000 μm.

The capillary tube may sequentially contact each of the heating blocks controlled at different temperatures once, repetitively twice, or more. The number of times that the capillary tube winds around the heating blocks varies depending on the kind of reaction, the accuracy, the product amount, the initial amount of the reaction sample, and etc.; however, may preferably range from 10 to 50 times, and, more preferably, from 20 to 30 times.

Hence, if the temperature of each heating block of the high-throughput device for performing continuous-flow reactions is set to the required temperature and a PCR mixture is injected into the capillary tube as a fluid, the PCR can be performed effectively by using the device.

Therefore, the present invention provides a high-throughput method of performing a continuous-flow nucleic acid amplification, comprising: (a) injecting at least one polymerase chain reaction mixture into the first open end of the capillary tube in the aforementioned device; and (b) controlling a flow rate of the polymerase chain reaction mixture at an appropriate speed and collecting a polymerase chain reaction product discharged from the second open end.

Generally, PCR is made up of three steps: (a) a melting step in which a double-stranded DNA (dsDNA) denatures into a single-stranded DNA (ssDNA); (b) an annealing step in which a designed primer binds to the single-stranded DNA; and (c) an extension step in which DNA is replicated from the position where the primer binds, thereby making double-stranded DNA. Also, there exist proper temperature and time conditions to perform the reaction of each step. These temperature and time conditions vary case-by-case depending on the base sequence of template DNA and primer, and the type of polymerase or catalyst. Specifically, it is preferred that the melting step is performed at 95˜100° C. for 1˜60 seconds, the annealing step is performed at 45˜65° C. for 1˜120 seconds, and the extension step is performed at 65˜72° C. for 30˜120 seconds.

In the method of amplifying nucleic acid, the temperature of each heating block of the high-throughput device for performing continuous-flow reactions is preferred to be set at the temperature for melting, annealing, and extension as mentioned above, and most preferably, approximately to 95° C., 60° C., and 72° C., respectively.

As the capillary tube sequentially or repetitively contacts the heating blocks for melting, annealing, and extension reaction, the DNA template injected into the capillary tube is amplified.

In the method of amplifying nucleic acid, the PCR cycle is determined by the number of times that the capillary tube repetitively contacts the heating blocks. The number of times varies case by case, but preferably 10 to 50 times, and more preferably, 20 to 30 times.

PCR mixtures contain reactants required to perform PCR, specifically, MgCl₂, dNTP (dATP, dCTP, dGTP, and dTTP) mixture, primer, thermophilic DNA polymerase, thermophilic DNA polymerase buffer, and template DNA. Further, for easy monitoring of a real-time PCR, the primer can be a molecular beacon, and the PCR mixtures may further comprise an intercalating dye.

The molecular beacon means a specially designed primer from which a fluorescent light is detected after the annealing step in PCR. The molecular beacon usually consists of dozens of nucleotides, and at both ends thereof, a fluorescent material and a quencher exist, respectively. In a free form, the molecular beacon has a hairpin structure, and the generation of fluorescence is inhibited because the fluorescent material and the quencher are close to each other. In contrast, if the molecular beacon is annealed to the template DNA at the annealing step in PCR, a fluorescent pigment on the molecular beacon emits fluorescent light because the distance between the fluorescent material and the quencher becomes long enough to overcome the inhibition of the quencher. The more PCR is performed, the more the amount of template DNA increases, thereby increasing the amount of the molecular beacon annealed to the template DNA. Therefore, the degree of DNA amplification can be measured in real-time in each cycle of the PCR by examining the level of fluorescent light using the molecular beacon.

The intercalating dye emits fluorescent light when it binds specifically to double-stranded DNA. Any intercalating dye well-known in the art, such as EtBr (Ethidium bromide) and SYBR GREEN™, may be used. The intercalating dye emits fluorescence when it binds specifically to double-stranded DNA amplified by PCR. It is, therefore, possible to estimate the amount of amplified product by measuring the intensity of the fluorescence signal.

In the method of amplifying nucleic acid in accordance with the present invention, it is preferred to use a syringe pump to inject a PCR mixture into the capillary tube and to control the flow rate of the PCR mixture. The PCR mixture moves from the first open end to the second open end by the syringe pump. The flow rate of the PCR mixture varies depending on the PCR reaction condition, and it can be adjusted in each reaction to obtain an optimum PCR result. Specifically, it is preferable that the flow rate of the PCR mixture injected into the capillary tube is in the range of 0.1 μl/min to 5 μl/min.

The PCR mixture can be injected into the capillary tube continuously or discontinuously. When PCR mixtures having different compositions are injected discontinuously, ‘carryover’ problem may arise. The ‘carryover’ means a phenomenon that a following sample is contaminated by the previous sample. To prevent this problem, it is preferred to separate each sample by air or an organic solvent that does not mix with samples, such as bromophenol blue. In addition, it is preferred to wash the remainder of the previous sample by injecting water or solvent such as buffer between the injection of PCR mixtures.

Hereinafter, specific aspects of the high-throughput device for performing continuous-flow reactions in accordance with the present invention will be described in detail, with reference to drawings.

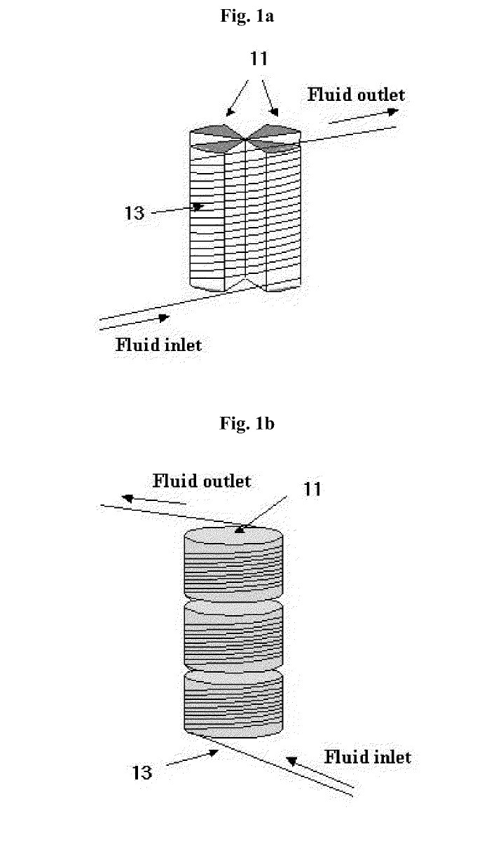

In accordance with a first preferred embodiment of the present invention, a high-throughput device for performing continuous-flow reactions can be prepared by winding a capillary tube 13 around at least two heating blocks 11 controlled at different temperatures. As shown FIG. 1a and FIG. 1b, the heating blocks 11 can be arranged in a serial or parallel mode. The capillary tube 13 contacts the heating blocks controlled at different temperatures by being wound around the heating blocks. As shown in FIG. 1a, in case the capillary tube 13 is wound around heating blocks 11 arranged in parallel, the fluid injected into the capillary tube undergoes reaction by passing sequentially or repetitively through heating blocks more than twice, controlled at different temperatures. On the other hand, as shown in FIG. 1b, in case the capillary tube 13 is wound around heating blocks 11 arranged in series, the injected fluid can undergo reaction by passing sequentially through heating blocks controlled at different temperatures.

Further, in accordance with a second preferred embodiment of the present invention, the high-throughput device for performing continuous-flow reactions may comprise an insulating block arranged to prevent the heating blocks from contacting each other for the efficient control of the temperature of each heating block.

For example, the present invention provides a high-throughput device for performing continuous-flow PCR comprising: (1) three solid heating blocks controlled at different temperatures; (2) an insulating block contacting the two adjacent heating blocks preventing them from contacting each other; and (3) a capillary tube having a first open end as an inlet for PCR mixture injection and a second open end as an outlet for the collection of the PCR product, to permit continuous flow of the PCR mixture from the first open end to the second open end, wherein the capillary tube contacts the three heating blocks sequentially or repetitively.

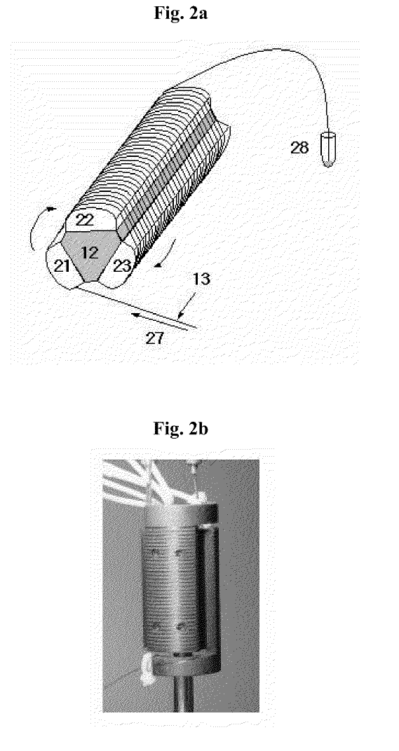

The second preferred embodiment of the high-throughput device for performing continuous-flow PCR is illustrated in FIG. 2a and FIG. 2b. FIG. 2a shows a schematic view of the device illustrating that the three heating blocks 21, 22, and 23 controlled at different temperatures are assembled with one insulating block 12, and a capillary tube is wound around the heating blocks. FIG. 2b shows a photograph of the device actually developed.

As mentioned above, the temperature of the heating blocks 21, 22, and 23 can be adjusted independently to the required temperatures suitable for each step of the PCR with an inserted heater and temperature controlling sensor in each of the heating block. The insulating block 12 is made of materials having very low heat conductivity to keep heating blocks at different temperatures. The PCR mixture 27 within the capillary tube 13 contacts sequentially or repetitively the heating blocks 21, 22, and 23 whose temperatures are set for melting, annealing, and extension reactions. As a result, a template DNA (nucleic acid) is amplified to produce a large amount of DNA 28.

Moreover, in accordance with a third preferred embodiment of a device having an insulating block, there is provided a high-throughput multiplex device for performing continuous-flow reactions, wherein at least two heating block-insulating block assemblies are assembled with at least two temperature-adjustable heating blocks to perform at least two independent reactions, and a capillary tube is wound on each assembly wherein the capillary tube has a first open end for fluid inlet and a second open end for fluid outlet to permit a continuous flow of a fluid from the first open end to the second open end.

In the high-throughput multiplex device, the number of the temperature-adjustable heating blocks may be two or more.

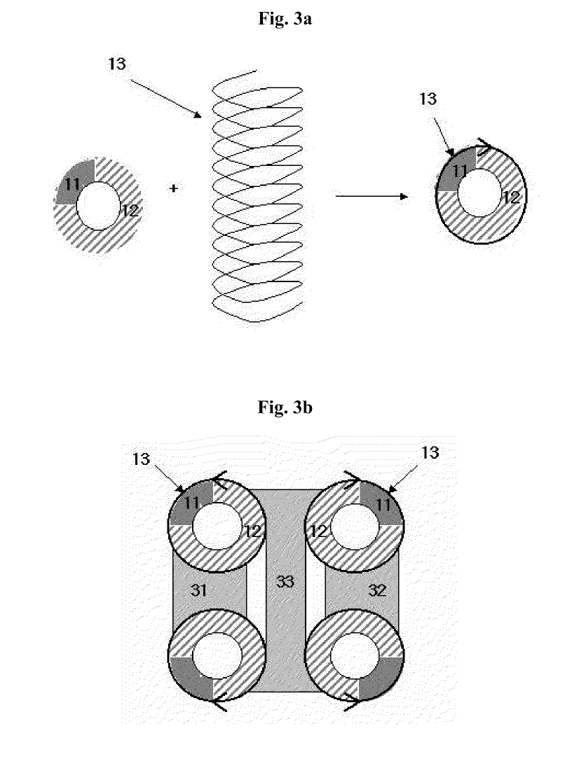

The third preferred embodiment of the multiplex device for performing continuous-flow reactions is illustrated in FIG. 3a to FIG. 3e. A method of preparing the multiplex device will now be described with reference to FIG. 3a. First, one heating block 11 is assembled with one insulating block 12 to prepare a heating block-insulating block assembly, and then the capillary tube 13 is wound around the heating block-insulating block assembly. Next, four heating block-insulating block assemblies around which a capillary tube is wound are respectively assembled with separate three temperature-adjustable heating blocks 31, 32, and 33, so that the three heating blocks 31, 32, and 33 contact at least two assemblies.

The plan view and front view of the multiplex device for performing continuous-flow reactions prepared by the method described above are shown in FIG. 3b and FIG. 3c, respectively. Also, the photograph of the multiplex device is shown in FIG. 3d and FIG. 3e.

The multiplex device performs four independent reactions at the same time. Seven heating blocks 11, 11, 11, 11, 31, 32, and 33 assembled to the multiplex device can be controlled at different temperatures for four independent operations. The capillary tube 13 wound around the heating block-insulating block assemblies contacts different heating blocks depending on its position. As shown in FIG. 3b, each capillary tube 13 contacts three heating blocks 11, 31, and 33 or 11, 32, and 33 repetitively controlled at different temperatures. The inside temperature of a capillary tube is controlled by the temperature of the heating block and influences the temperature of fluids flowing within the capillary tube, so that the fluids pass through three different temperature zones repetitively.

The use of the multiplex device offers an advantage that four independent reactions can be performed within four independent capillary tubes at the same time.

Specifically, if a PCR mixture for DNA amplification is used as a fluid flowing within the capillary tube, the multiplex device for performing continuous-flow reactions can be used for PCR. The method of performing PCR is similar to the aforementioned method in the device for performing PCR (FIG. 3c). That is, a PCR mixture 27 within a capillary tube 13 repetitively contacts heating blocks whose temperatures are set for melting 33, annealing 11, and extension 31 and 32. As a result, a template DNA (nucleic acid) is amplified to produce a large amount of DNA 28.

The heating block 11 performing the annealing step of PCR has an optimum annealing temperature depending on samples. The optimum annealing temperature varies in each PCR depending on the base sequence of a primer and a template DNA and is preferably set in the range of approximately 45° C. to 65° C. The heating block 33 performing the melting step of PCR contacts four heating block-insulating block assemblies around which the capillary tubes are wound. It is preferable for the temperature of the heating block 33 to be set approximately at 95° C. The heating blocks 31 and 32 performing the extension step of PCR contact two heating block-insulating block assemblies around which the capillary tubes are wound. The temperature of the heating blocks 31 and 32 is determined depending on the DNA polymerase, but is preferably set at 72° C. when Taq polymerase is used.

In addition, in order to monitor the degree of DNA amplification in real-time during PCR, a real-time detection apparatus may be employed.

Specifically, there is provided a high-throughput device for performing continuous-flow reactions, which detects the degree of real-time reaction, further comprising: (a) a fluorescence-inducing apparatus having a light source for inducing fluorescence, a unit for detecting fluorescence, and an optical system for collecting emitted fluorescence to the unit for detecting fluorescence after light irradiation to the capillary tube; and (b) a scanning unit changing the relative positions of the fluorescence-inducing apparatus and the capillary tube.

A laser or a lamp irradiating a light with specific wavelength can be used as the light source for inducing fluorescence and a PMT or a diode can be used as the fluorescence detecting unit. The optical system may comprise a dichromatic mirror to pass and reflect the laser light and an object lens to focus the laser light on the capillary tube, collect the fluorescent light generated from the capillary tube, and transfer it to the dichromatic mirror. On the other hand, the scanning unit functions to change the relative positions of the fluorescence-inducing apparatus and the capillary tube by moving the capillary tube-wound heating block back and forth at a constant speed when the fluorescence-inducing apparatus is fixed, or moving the fluorescence-inducing apparatus back and forth at a constant speed when the heating block is fixed.

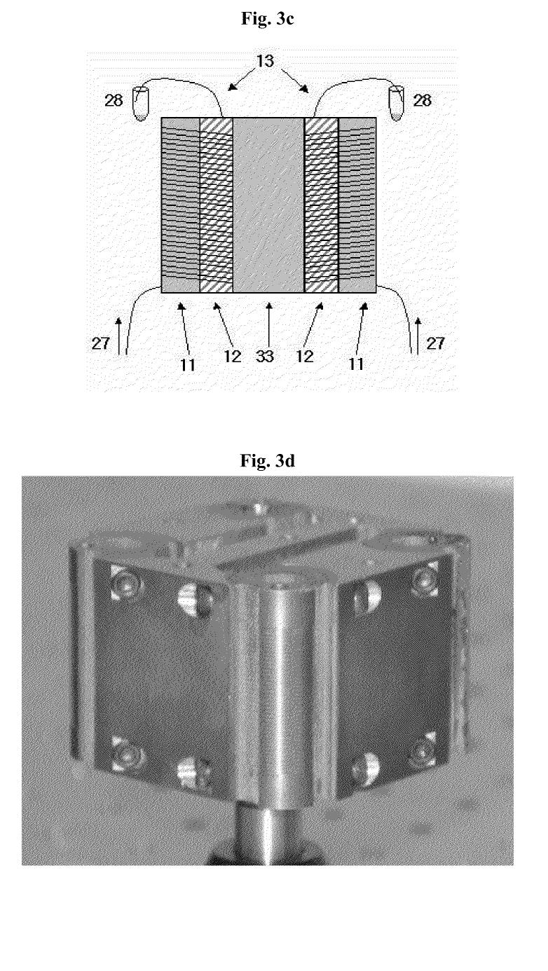

Referring to FIG. 4, a method for detecting the degree of DNA amplification in real-time PCR is described. A PCR mixture 27 containing a material that can emit fluorescence as DNA is amplified is injected into the capillary tube 13. Subsequently, a laser light 41 with a specific wavelength is irradiated to the capillary tube 13 through a dichromatic mirror 43 and an object lens 44. The amount of fluorescence 42 emitted from the capillary tube is measured by a unit for detecting fluorescence to measure the degree of DNA amplification within the capillary tube in real-time.

The high-throughput device for performing continuous-flow reactions according to the present invention is useful for reacting continuous-flow fluids, especially, for performing the polymerase chain reaction (PCR). Further, the high-throughput multiplex device according to the present invention provides the facility to perform at least two independent reactions having different reaction conditions simultaneously. Accordingly, the device according to the present invention is more advantageous for the construction of a DNA multiplex amplification device which can be smaller in size and portable. Because the size of the wound capillary tube is similar to that of micro channels on biochips, the device can be easily integrated with lab-on-a-chip. In addition, the degree of DNA amplification during PCR can be monitored in real-time by coupling with a real-time detection apparatus.

The following Examples are intended to further illustrate the present invention without limiting its scope.

### Example 1

### Construction of a Device for Performing Continuous-Flow Reactions

### (1-1) Construction of a Device for Performing Continuous-Flow PCR

In the device for performing continuous-flow PCR according to the present invention as shown in FIG. 2a, the three heating blocks 21, 22, and 23 were prepared with copper and an insulating block 12 was prepared with bakelite.

The three heating blocks were mounted on each side of the insulating block forming a heating block-insulating block assembly with 30 mm in diameter and 65 mm in height (FIG. 2b). The heating block-insulating block assembly has the insulating block inside and the three heating blocks with an arc of same length that surround the insulating block.

Each of these heating blocks provides holes for inserting the heater and the temperature sensor for measuring and controlling the temperature of the heating block. Specifically, the hole for heater has 3.1 mm in diameter with 32 mm in length (Firerod, Watlow, St. Louis, Mo.) while the hole for temperature sensor has 1 mm in diameter with 27 mm in length (Watlow, St. Louis, Mo.).

A helical groove of 250 μm in depth and 250 μm in width was formed on the surface of the heating block-insulating block assembly with 1.5 mm pitch per turn of the helix. This helical groove functions to fix the position of a capillary tube around the heating blocks and to facilitate the efficient heat transfer in reaction. The helical groove was formed in the vertical direction of the heating block-insulating block assembly in 33 rotations, which correspond to the number of the PCR cycles in DNA amplification reaction. Total approximately 3.5 meter of the capillary tube was used encompassing parts required for solution injection and solution collection and parts for helical groove.

The capillary tube winding the beginning of the heating block for the melting step and the ending of the heating block for the extension step were elongated to help a complete PCR cycle from the initial melting to final extension steps, respectively.

The capillary tube is protruded at both ends of the heating blocks in the heating block-insulating block assembly as shown in FIG. 2a and FIG. 2b.

A fused silica capillary tube coated with polyimide having 240 μm in the outer diameter and 100 μm in the inner diameter was used (Polymicro Technologies, Phoenix, Ariz.). To prevent the adsorption of biomolecules such as DNA and protein, etc. on the inner wall of the capillary tube, the inner wall of the capillary tube was silanized. For silanization initially the capillary tubes were flushed with methanol for 30 minutes, dried at 40° C. for 12 hours, and then kept filled with a DMF (dimethylformamide) solution containing 0.02M TMS (trimethylchlorosilane) and 0.04M imidazole at room temperature for a day. When the silanization reaction was completed, the capillary tubes were rinsed with methanol and then with sterilized water.

The device for performing continuous-flow PCR was prepared by fitting the capillary tubes into the helical groove formed on the surface of the heating block-insulating block assembly.

### (1-2) Construction of a Multiplex Device for Performing Continuous-Flow PCR

As shown in FIG. 3b to FIG. 3e, a multiplex device for performing continuous-flow PCR was prepared. Like Example (1-1), copper and bakelite were used to prepare heating blocks and insulating blocks, respectively.

First, one heating block 11 was assembled with one insulating block 12 to prepare a heating block-insulating block assembly with 20 mm in diameter and 40 mm in height. Four of such heating block-insulating block assemblies were prepared. A helical groove of 240 μm in depth and 240 μm in width was formed on the surface of each heating block-insulating block assembly with 1 mm pitch per turn of the helix. The helical groove was formed in the vertical direction of the heating block-insulating block assembly in 34 rotations. Total approximately 2 meter of the capillary tube was used encompassing parts required for solution injection and solution collection and parts for helical groove.

Like Example (1-1), the holes for inserting a heater and a temperature sensor were formed on each heating block of the heating block-insulating block assembly. The fused silica capillary tube used in Example (1-1) or PTFE capillary tube (Cole-Parmer Instrument, Co.) was used.

Four heating block-insulating block assemblies around which capillary tubes had been wound were assembled with three separate heating blocks 31, 32, and 33 so that two heating blocks 31 and 32 contacted two capillary tubes and one heating block 33 contacted four capillary tubes, resulting a multiplex device for performing continuous-flow PCR (FIG. 3b, FIG. 3d, and FIG. 3e).

### Example 2

### Continuous-Flow PCR

PCR was performed with a PCR mixture solution flowing continuously within the capillary tube in the device prepared in Example (1-1).

A plasmid DNA isolated from bacterial kanamycin resistance gene was used as a template DNA for amplifying a 323 bp fragment thereof while using primers represented by SEQ ID NO:1 and SEQ ID NO:2. The PCR mixture solution (total 50 μL) has the following composition: 3 μL of 25 mM MgCl₂, 5 μL of 10× thermophilic DNA polymerase buffer (500 mM KCl, 100 mM Tris-HCl, 1% Triton® X-100), 1 μL of 10 mM PCR nucleotide mixture (dATP, dCTP, dGTP, and dTTP in water (10 mM each)), 3.3 μL of 12 μM upstream primer, 3.3 μL of 12 μM downstream primer, 0.25 μL of 5 unit/μL Taq DNA polymerase, 1 μL (1 ng) of template DNA, and 33.15 μL of sterilized distilled water.

A syringe pump (22 Multiple Syringe Pump, Harvard Apparatus) was used to inject the PCR mixture into the capillary tube continuously at the flow rate in the range from 0.3 μL/min to 5.0 μL/min. A gas tight syringe (250 μL capacity) filled with the PCR mixture was connected to the pump. By pumping, the PCR mixture in the syringe was injected into the capillary tube whose end for fluid inlet (at the beginning of the heating block for melting reaction) was connected to the end of the syringe, thereby performing continuous flow.

The temperature of each heating block of the device was maintained at 95° C., 60° C., and 72° C., respectively, and the PCR mixture contacted the heating blocks repetitively. PCR was performed at various flow rates, specifically, at 0.3, 0.5, 1.0, 3.0, and 5.0 μL/min, respectively.

The PCR product was collected from a fluid outlet end of the capillary tube (at the end of the heating block for extension reaction) in 90 minutes after the injection of the PCR mixture when the flow rate was 0.3 μL/min, and in 5 minutes when the flow rate was 5.0 μL/min, respectively.

### Example 3

### Identification of Amplified DNA

Gel electrophoresis was performed in order to identify the DNA amplification of the PCR mixture. 10 μL of the PCR product collected in Example 2 was analyzed by 2% agarose gel electrophoresis in TBE buffer. In order to check the level of DNA amplification, a sample for a positive control amplified by a commercial machine (MBS 0.2 G, Hybaid, U.K.) and a size marker were loaded together. The PCR in the commercial machine was initiated at 95° C. for 2 minutes, and the subsequent cycles were performed at 95° C. for 30 seconds, 60° C. for 1 minute, and 72° C. for 2 minutes. These cycles were repeated 33 times, and then the product was kept at 72° C. for 5 minutes. The PCR reaction was concluded by cooling the PCR product to 4° C.

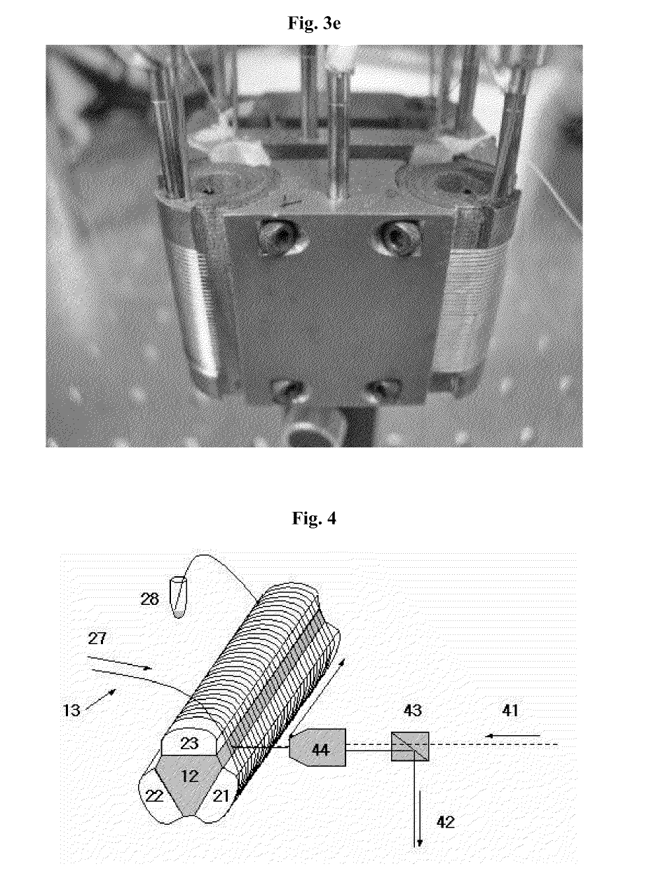

FIG. 5 shows the results from gel electrophoresis of PCR products. In FIG. 5, lane 1 (positive control) shows the result of DNA amplification performed using the commercial machine, lanes 2 to 6 show the difference of DNA amplification level at various flow rates ranging from 0.3 μL/min to 5.0 μL/min (from the left, 0.3, 0.5, 1.0, 3.0, and 5.0μL/min, respectively), lane 7 (negative control) shows the DNA not amplified by the PCR, and lane 8 shows size markers to measure the size of amplified DNA. As shown in FIG. 5, the results clearly showed that high efficiency of DNA amplification could be achieved by using the device according to the present invention. In particular, the results showed that the slower the flow rate was, the higher the amplification efficiency was since the extension was fully performed when the flow rate was slow.

### Example 4

### Sequential DNA Amplifications with Different PCR Mixtures

The present inventors investigated whether the device for performing continuous-flow PCR according to the present invention can be used to perform DNA amplifications for each template DNA when PCR mixtures having different compositions were injected sequentially.

PCR mixtures containing four different DNA templates and a pair of primers for each DNA template were prepared to perform the aforementioned PCR scheme. The used DNA templates and primers are described in Table 1 below.

TABLE 1

| Sample | | | |
|---|---|---|---|
| No. | Template DNA | Primers | Source |
| 1 | Lambda DNA | SEQ ID NO: 1 and SEQ ID NO: 2 | Promega |
| | | (designed to amplify 500 bp | |
| | | fragment of the template DNA) | |
| 2 | A plasmid DNA | SEQ ID NO: 3 and SEQ ID NO: 4 | Takara |
| | isolated from | (designed to amplify 323 bp | |
| | bacterial kanamycin | fragment of the template DNA) | |
| | resistance gene | | |
| 3 | PCS2HA/LM04 | SEQ ID NO: 5 and SEQ ID NO: 6 | Postech |
| | | (designed to amplify 497 bp | Univ., |
| | | fragment of the template DNA) | laboratory |
| 4 | Lhx3-LIM1 | SEQ ID NO: 7 and SEQ ID NO: 8 | of |
| | | (designed to amplify 267 bp | Department |
| | | fragment of the template DNA) | Life |
| | | | Science |

PCR mixtures (sample 1 to 4) including each template DNA and a pair of primers thereof were prepared. The composition of each PCR mixture was identical to that used in Example 2. Samples were injected repeatedly in the following order: sample 1 (2 μL)-air gap (<1 cm) -bromophenol blue (2 μL)-air gap (<1 cm)-sample 2 (2 μL)-air gap (<1 cm)-bromophenol blue (2 μL)-air gap (<1 cm)-sample 3 (2 μL)-air gap (<1 cm)-bromophenol blue (2 μL)-air gap (<1 cm)-sample 4 (24)-air gap (<1 cm)-bromophenol blue (2 μL)-air gap (<1 cm)-sample 1 (2 μL).

The air gap and bromophenol blue buffer (30% glycerol, 30 mM EDTA, 0.03% bromophenol blue, 0.03% xylene cyanol) (Takara) were injected between each PCR mixture in order to prevent carryover.

Subsequently, each PCR product was collected separately at the end of the fluid outlet of the capillary tube by the color of the bromophenol blue buffer and the presence of air gap.

Besides, to check the effects of the inner wall coating on the efficiency of DNA amplifications, the present inventors performed PCR using a capillary tube whose inner wall was coated with trimethylchlorosilane (TMS) and a uncoated capillary tube, respectively.

Gel electrophoresis was performed according to the same procedure as Example 3 to identify the DNA amplification of the PCR product. In addition, to check the level of DNA amplification, a sample for a positive control amplified by a commercial machine (MBS 0.2 G, Hybaid, U.K.) and a size marker were loaded together. The PCR in the commercial machine was initiated at 95° C. for 2 minutes, and the subsequent cycles were performed at 95° C. for 30 seconds, 60° C. for 1 minute, and 72° C. for 2 minutes. These cycles were repeated 33 times, and then the product was kept at 72° C. for 5 minutes. The PCR reaction was concluded by cooling the PCR product to 4° C.

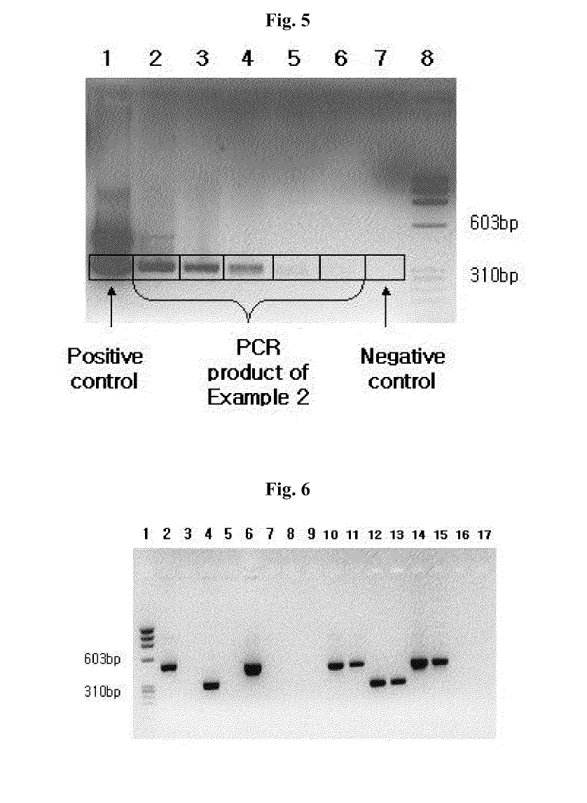

FIG. 6 shows the results from gel electrophoresis of PCR products. In FIG. 6, lane 1 shows size markers to measure the size of amplified DNA and lanes 2, 4, 6, 8, 10, 12, 14, and 16 show the result of DNA amplification for samples 1 to 4 performed using commercial PCR machines. Further, lanes 3, 5, 7, and 9 show the result of DNA amplification for samples 1 to 4 using the uncoated capillary tube, and lanes 11, 13, 15, and 17 show the result of DNA amplification for samples 1 to 4 using the capillary tube whose inner wall was coated with TMS.

As shown in lanes 11, 13, and 15 of FIG. 6, it was found that the DNA amplifications for samples 1 to 3 were performed efficiently. As a result, the present device can be applied to perform sequential DNA amplifications with different PCR mixtures.

While the invention has been described with respect to the above specific embodiments, it should be recognized that various modifications and changes may be made to the invention by those skilled in the art which also fall within the scope of the invention as defined by the appended claims.

What is claimed is:

1. A high-throughput device for performing a continuous-flow reaction comprising:
(1) at least two solid heating blocks controlled at different temperatures; and
(2) at least one capillary tube having a first open end for fluid inlet and a second open end for fluid outlet to permit a continuous flow of a fluid from the first open end to the second open end, wherein the capillary tube contacts the heating blocks sequentially or repetitively.

2. A high-throughput device for performing a continuous-flow reaction comprising:
(1) at least two solid heating blocks controlled at different temperatures;
(2) at least one insulating block contacting the heating blocks and arranged to prevent the heating blocks from contacting each other; and
(3) at least one capillary tube having a first open end for fluid inlet and a second open end for fluid outlet to permit a continuous flow of a fluid from the first open end to the second open end, wherein the capillary tube contacts the heating blocks sequentially or repetitively.

3. The device of claim 1 or 2, wherein the device performs a polymerase chain reaction.

4. The device of claim 1 or 2, wherein the heating blocks are controlled at different temperatures by a heater and a temperature sensor.

5. The device of claim 1 or 2, wherein the heating blocks are made of a heat conductive metal selected from the group consisting of copper, iron, aluminum, brass, gold, silver, and platinum.

6. The device of claim 2, wherein the insulating block is made of bakelite or an acrylic polymer resin.

7. The device of claim 1 or 2, wherein the capillary tube is made of a material selected from the group consisting of glass, fused silica, polytetrafluoroethylene, and polyethylene.

8. The device of claim 1 or 2, wherein the outer wall of the capillary tube is coated with polyimide or polytetrafluoroethylene.

9. The device of claim 1 or 2, wherein the inner wall of the capillary tube is coated with at least one material selected from the group consisting of trimethylchlorosilane, dimethyldichlorosilane, methyltrichlorosilane, trimethylmethoxysilane, dimethyldimethoxysilane, and methyltrimethoxysilane.

10. The device of claim 1 or 2, wherein the capillary tube is wound on the outer surface of the heating blocks.

11. The device of claim 10, wherein the capillary tube is fit into a helical groove formed on the outer surface of the heating blocks.

12. The device of claim 10, wherein the capillary tube is wound 10 to 50 times.

13. The device of claim 2, which performs a polymerase chain reaction, comprising:
(1) three solid heating blocks controlled at different temperatures;
(2) an insulating block contacting the heating blocks and arranged to prevent the heating blocks from contacting each other; and
(3) a capillary tube having a first open end for an inlet of a polymerase chain reaction mixture and a second open end for an outlet of the polymerase chain reaction mixture, to permit continuous flow of the polymerase chain reaction mixture from the first open end to the second open end, wherein the capillary tube contacts the three heating blocks sequentially or repetitively.

14. The device of claim 1 or 2, which detects the degree of the reaction in real-time, further comprising:
(a) a fluorescence-inducing apparatus having a light source for inducing fluorescence, a unit for detecting fluorescence, and an optical system for collecting emitted fluorescence to the unit for detecting fluorescence after light irradiation to the capillary tube; and
(b) a scanning unit changing the relative positions of the fluorescence-inducing apparatus and the capillary tube.

15. The device of claim 14, wherein the reaction is a polymerase chain reaction.

16. A high-throughput multiplex device for performing continuous-flow reactions, wherein at least two heating block-insulating block assemblies are assembled with at least two temperature-adjustable heating blocks to perform at least two independent reactions, and a capillary tube is wound on each assembly wherein the capillary tube has a first open end for fluid inlet and a second open end for fluid outlet to permit a continuous flow of a fluid from the first open end to the second open end.

17. A high-throughput method of performing a continuous-flow nucleic acid amplification, comprising the steps of:
(a) injecting at least one polymerase chain reaction mixture into the first open end of the capillary tube of the device of claim 1 or 2; and
(b) controlling the flow rate of the polymerase chain reaction mixture at an appropriate speed and collecting a polymerase chain reaction product discharged from the second open end.

18. The method of claim 17, wherein the number of solid heating blocks of the device of claim 1 or 2 is three, and the capillary tube contacts sequentially or repetitively the heating blocks each of whose temperature is set at 95˜100° C., 45˜65° C., and 65˜72° C.

19. The method of claim 17, wherein the capillary tube repetitively contacts the heating blocks 10 to 50 times.

20. The method of claim 17, wherein the polymerase chain reaction mixture comprises MgCl₂, dNTP mixture, at least one primer, at least one thermophilic DNA polymerase, a thermophilic DNA polymerase buffer, and at least one template DNA.

21. The method of claim 20, wherein the primer is a molecular beacon.

22. The method of claim 20, wherein the polymerase chain reaction mixture further comprises at least one intercalating dye that emits fluorescence when intercalated into double-stranded DNA.

23. The method of claim 17, wherein the polymerase chain reaction mixture moves from the first open end to the second open end by a pump.

24. The method of claim 17, wherein the polymerase chain reaction mixture is injected continuously or discontinuously in step (a).

25. The method of claim 24, wherein when polymerase chain reaction mixture is injected discontinuously in different compositions each other, an organic solvent or air is injected between injections.
